(19) United States
(12) Patent Application Publication
(10) Pub. No.: US 2006/0089384 A1
(43) Pub. Date: Apr. 27, 2006

(54) Ophthalmic compositions and methods of using the same

(76) Inventors: George E. Minno, Suwanee, GA (US); Susan Caballa, Sugar Hill, GA (US)

Correspondence Address: HUTCHISON LAW GROUP PLLC, PO BOX 31686, RALEIGH, NC, 27612, US

(21) Appl. No.: 10/972,571

(22) Filed: Oct. 25, 2004

(51) Int. Cl. A61K 31/453

(52) U.S. Cl. 514/320

(57) ABSTRACT

Ophthalmic compositions are provided that comprise or consist essentially of (a) ketotifen or a ketotifen salt, (b) a non-ionic tonicity agent, and (c) water. The concentration of ketotifen or the ketotifen salt is preferably from 0.01% to 0.05%. The non-ionic tonicity agent is preferably glycerol and the concentration of the glycerol is preferably from 4% to 7%. The compositions preferably have an osmolality from 400 to 750 milliosmoles/Kg. Methods of treating allergic conjunctivitis using the ophthalmic compositions are also provided.

### FIELD

The invention generally relates to ophthalmic compositions containing ketotifen and/or a ketotifen salt and methods of using the same.

### BACKGROUND

Various ophthalmic compositions are known for treating allergic conjunctivitis. For example, U.S. Pat. No. 6,274,626 is directed towards compositions comprising the antihistamine pheniramine in combination with povidone for preventing and treating ophthalmic allergic responses. Solutions according to U.S. Pat. No. 6,274,626 may contain buffers, various surfactants, stabilizers, isotonic agents and the like which aid in making ophthalmic compositions more comfortable to the user. The aqueous solutions of U.S. Pat. No. 6,274,626 are typically adjusted with tonicity agents to approximate the osmotic pressure of normal lachrymal fluids, which is stated to be equivalent to a 0.9% solution of sodium chloride or a 2.5% solution of glycerol. An osmolality of about 225 to 400 mOsm/kg is preferred for the solutions, and is more preferably 280 to 320 mOsm/kg. U.S. Pat. No. 6,274,626 also states that excess salt or other tonicity agent may result in the formation of a hypertonic solution that will cause stinging and eye irritation.

Ophthalmic compositions for treating allergic conjunctivitis that contain ketotifen are also known. For example, U.S. Pat. Nos. 6,774,137 and 6,777,429 relate to an ophthalmic composition comprising ketotifen as a pharmaceutically active agent, comprising a ketotifen salt in a concentration of 0.01 to 0.04%, a non-ionic tonicity agent in an amount such that the total tonicity of the composition has an osmolarity in the range of 210 to 290 milliosmoles, optionally a preservative, an acid or base for bringing the pH to weak acidity, and water. The patents disclose that the ophthalmic composition can be used for the treatment and the temporary prevention of itching of the eye due to allergic conjunctivitis. The patents also disclose that glycerol is the preferred non-ionic tonicity agent and that if glycerol is used, the concentration is preferably in the range of 1.5 to 2.5%.

One commercially available product for temporary prevention of itching of the eye due to allergic conjunctivitis, Zaditor™ ketotifen fumarate ophthalmic solution, is a sterile ophthalmic solution containing 0.0345% ketotifen fumarate (equivalent to 0.025% ketotifen), 0.01% benzalkonium chloride, glycerol, sodium hydroxide/hydrochloric acid (to adjust pH), and purified water. The product has a pH of 4.4 to 5.8 and an osmolality of 210-300 mOsm/kg.

### SUMMARY

In one aspect, an ophthalmic composition is provided that consists essentially of (a) ketotifen or a ketotifen salt in a concentration of 0.01% to 0.05%; (b) a non-ionic tonicity agent in a concentration such that the composition has an osmolality from 400 to 750 milliosmoles/Kg; and (c) water.

In another aspect, an ophthalmic composition is provided that consists essentially of (a) ketotifen or a ketotifen salt in a concentration of 0.01% to 0.05%; (b) glycerol in a concentration of 3.5% to 7%; and (c) water.

In yet another aspect, an ophthalmic composition is provided that consists essentially of (a) ketotifen or a ketotifen salt in a concentration of 0.01% to 0.05%; (b) glycerol in a concentration of greater than 3.5% such that the composition has an osmolality from 400 to 750 milliosmoles/Kg; and (c) water.

In a further aspect, an ophthalmic composition is provided that consists essentially of (a) ketotifen fumarate in a concentration of 0.0345%; (b) glycerol in a concentration of 5.75% to 6.25%; (c) benzalkonium chloride in a concentration of 0.01%; and (d) water. The pH of the composition is from 4.4 to 5.8.

In another aspect, a method of treating allergic conjunctivitis is provided comprising administering to a subject suffering from or susceptible to allergic conjunctivitis an effective amount of an ophthalmic composition consisting essentially of (a) ketotifen or a ketotifen salt in a concentration of 0.01% to 0.05%; (b) a non-ionic tonicity agent in a concentration such that the composition has an osmolality from 400 to 750 milliosmoles/Kg; and (c) water.

In yet another aspect, a method of treating allergic conjunctivitis is provided comprising administering to a subject suffering from or susceptible to allergic conjunctivitis an effective amount of an ophthalmic composition consisting essentially of (a) ketotifen or a ketotifen salt in a concentration of 0.01% to 0.05%; (b) glycerol in a concentration of 3.5% to 7%; and (c) water.

In a further aspect, a method of treating allergic conjunctivitis is provided comprising administering to a subject suffering from or susceptible to allergic conjunctivitis an effective amount of an ophthalmic composition consisting essentially of (a) ketotifen or a ketotifen salt in a concentration of 0.01% to 0.05%; (b) glycerol in a concentration of greater than 3.5% such that the composition has an osmolality from 400 to 750 milliosmoles/Kg; and (c) water.

In another aspect, a method of treating allergic conjunctivitis is provided comprising administering to a subject suffering from or susceptible to allergic conjunctivitis an effective amount of an ophthalmic composition consisting essentially of (a) ketotifen fumarate in a concentration of 0.0345%, (b) glycerol in a concentration of 5.75% to 6.25%, (c) benzalkonium chloride in a concentration of 0.01%, and (d) water. The pH of the composition is from 4.4 to 5.8.

In yet another aspect, an ophthalmic composition is provided that comprises (a) ketotifen or a ketotifen salt in a concentration of 0.01% to 0.05%; (b) a non-ionic tonicity agent in a concentration such that the composition has an osmolality from 400 to 750 milliosmoles/Kg; and (c) water.

In a further aspect, an ophthalmic composition is provided that comprises (a) ketotifen or a ketotifen salt in a concentration of 0.01% to 0.05%; (b) glycerol in a concentration of 3.5% to 7%; and (c) water.

In yet a further aspect, an ophthalmic composition is provided that comprises (a) ketotifen or a ketotifen salt in a concentration of 0.01% to 0.05%; (b) glycerol in a concentration of greater than 3.5% such that the composition has an osmolality from 400 to 750 milliosmoles/Kg; and (c) water.

In another aspect, an ophthalmic composition is provided that comprises (a) ketotifen fumarate in a concentration of 0.0345%; (b) glycerol in a concentration of 5.75% to 6.25%; (c) benzalkonium chloride in a concentration of 0.01%; and (d) water. The pH of the composition is from 4.4 to 5.8.

### DETAILED DESCRIPTION

The present invention relates to ophthalmic compositions containing ketotifen and/or a ketotifen salt as well as methods of using the same.

Conventional aqueous ophthalmic solutions are typically adjusted with tonicity agents to approximate the osmotic pressure of normal lachrymal fluids, which, as stated in U.S. Pat. No. 6,274,626, is equivalent to a 2.5% solution of glycerol. Excess tonicity agent is typically thought, as also stated in U.S. Pat. No. 6,274,626, to result in the formation of a hypertonic solution that will cause stinging and eye irritation. Surprisingly, it has been discovered that increasing the osmolality of and/or increasing the concentration of glycerol in ophthalmic compositions containing ketotifen (or a salt thereof) results in greater comfort, a cooling sensation, and/or less stinging, burning, or irritation due to the ophthalmic composition.

The ophthalmic compositions of the present invention comprise ketotifen or a ketotifen salt, a non-ionic tonicity agent, and water. In some embodiments, the ophthalmic compositions consist essentially of ketotifen or a ketotifen salt, a non-ionic tonicity agent, and water. The compositions may include a preservative and may also include an acid or base to adjust the pH of the composition.

The ketotifen or ketotifen salt is present in the composition in a concentration of 0.01% to 0.05%, preferably 0.01% to 0.04%, more preferably 0.02% to 0.03% (as used herein, “concentration” of a component of an ophthalmic composition means concentration based on mass of the component per total volume of the composition (i.e., g/mL), and is typically expressed as a percentage). Any ophthalmically acceptable ketotifen salt may be used, although ketotifen fumarate is preferred. Ketotifen fumarate is represented by the following formula: 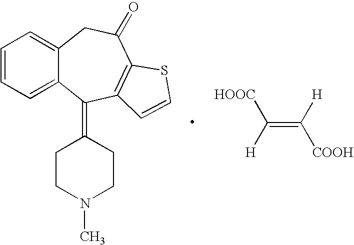
In some embodiments, the ketotifen or ketotifen salt is provided in a concentration such that the concentration of ketotifen base in the composition is 0.02% to 0.03%, preferably 0.0225% to 0.0275%, more preferably 0.025%. Concentrations of ketotifen salts yielding such concentrations of ketotifen base may be readily calculated; for example, using ketotifen fumarate in a concentration of 0.0345% in the composition provides a concentration of ketotifen base in the composition of 0.025%.

The non-ionic tonicity agent is preferably glycerol, although other non-ionic tonicity agents may be used such as, for example, urea, sorbitol, mannitol, propylene glycol, and dextrose. In some embodiments, the non-ionic tonicity agent is provided in a concentration such that the composition has an osmolality from 400 to 750 milliosmoles/kilogram (mOsm/Kg), preferably from 425 to 700 mOsm/Kg, more preferably from 550 to 700 mOsm/Kg, even more preferably from 600 to 700 mOsm/Kg, and yet even more preferably from 650 to 700 mOsm/Kg. In other embodiments, glycerol is used as the non-ionic tonicity agent in a concentration of from 3.5% to 7%, preferably from 4.5% to 7%, more preferably from 5% to 7%, even more preferably from 5.5. % to 6.5%, and yet even more preferably from 5.75% to 6.25%. In further embodiments, glycerol is used as the non-ionic tonicity agent in a concentration of greater than 3.5%, preferably greater than 4.5%, more preferably greater than 5%, and even more preferably greater than 5.5%. In yet other embodiments, glycerol is used as the non-ionic tonicity agent in a concentration of greater than 3.5%, preferably greater than 4.5%, more preferably greater than 5.5%, even more preferably from 5% to 7%, and yet even more preferably from 5.5% to 6.5%, such that the composition has an osmolality from 400 to 750 mOsm/Kg, preferably from 425 to 700 mOsm/Kg, more preferably from 550 to 700 mOsm/Kg, even more preferably from 600 to 700 mOsm/Kg, and yet even more preferably from 650 to 700 mOsm/Kg.

As stated above, the compositions may include a preservative. A preservative is preferred when the composition is packaged for multidose units, but may be absent from the composition if desired (e.g., in single dose units of the composition). Any preservative may be used with the compositions, but benzalkonium chloride is preferred. Other preservatives that may be used include Polyquad preservative (Alcon); perborate (e.g., sodium perborate from Ciba); Purite preservative (stabilized chlorine dioxide) (Allergan); other quaternary ammonium compounds such as benzoxonium chloride; alkyl-mercury salts of thiosalicylic acid such as, for example, thiomersal, phenylmercuric nitrate, phenylmercuric acetate, and phenylmercuric borate; parabens such as, for example, methylparaben or propylparaben; alcohols such as, for example, chlorobutanol, benzyl alcohol, and phenyl ethanol; guanidine derivatives such as, for example, chlorhexidine or polyhexamethylene biguanide; and the like. When a preservative is used in the composition, the preservative is typically provided in a concentration of 0.005% to 0.02%, preferably 0.01%, although other concentrations may be used.

The ophthalmic compositions typically have a pH from 4 to 6, preferably from 4.4. to 5.8, although the compositions may also have a pH outside of these ranges. An acid or base may be added to adjust the pH of the compositions to the desired level. Typically, only small amounts of an acid or base will be needed to adjust the pH of the composition. The preferred acid and base for adjusting the pH are hydrochloric acid and sodium hydroxide.

In some embodiments, the compositions are free or substantially free of stabilizers such as ethylene diamine tetraacetic acid (EDTA) and salts thereof, Dequest, and Desferal (e.g., as used in compositions described in U.S. Pat. Nos. 6,776,982 and 6,468,548); polymers comprising chitosan (e.g., as used in compositions described in U.S. Patent Application No. 2003/0031718); linear polysaccharide compounds such as hyaluronic acid compounds (e.g., as used in compositions described in International Publication No. WO 02/100437); biocompatible polymers/thickeners such as polyoxyethylene-polyoxypropylene copolymers and acrylic acid homo- and co-polymers (e.g., as used in compositions described in International Publication No. WO 02/100436); antioxidants; and/or active agents other than ketotifen. Preferably, the compositions consisting essentially of ketotifen or a ketotifen salt, a non-ionic tonicity agent, and water are free or substantially free of these components.

As stated above, in some embodiments, the ophthalmic composition comprises ketotifen or a ketotifen salt, a non-ionic tonicity agent, and water, and optionally includes a preservative and/or an acid or base to adjust the pH of the composition. In other embodiments, the ophthalmic composition consists essentially of ketotifen or a ketotifen salt, a non-ionic tonicity agent, and water, and optionally includes a preservative and/or an acid or base to adjust the pH of the composition. In further embodiments, the composition consists of ketotifen or a ketotifen salt, a non-ionic tonicity agent, and water, and optionally includes a preservative and/or an acid or base to adjust the pH of the composition. In yet other embodiments, the composition consists of ketotifen or a ketotifen salt, a non-ionic tonicity agent, and water.

In one particularly preferred embodiment, the ophthalmic composition consists essentially of ketotifen fumarate in a concentration of 0.0345%, glycerol in a concentration of 5.75% to 6.25%, benzalkonium chloride in a concentration of 0.01%, and water. The pH of such a composition is preferably from 4.4. to 5.8, and the osmolality of such a composition is preferably from 625 to 750 mOsm/Kg, more preferably from 650 to 725 mOsm/Kg.

In another embodiment, the ophthalmic composition comprises ketotifen fumarate in a concentration of 0.0345%, glycerol in a concentration of 5.75% to 6.25%, benzalkonium chloride in a concentration of 0.01%, and water. The pH of such a composition is preferably from 4.4. to 5.8, and the osmolality of such a composition is preferably from 625 to 750 mOsm/Kg, more preferably from 650 to 725 mOsm/Kg.

The ophthalmic compositions are useful for the treatment and temporary prevention of the signs and symptoms of allergic conjunctivitis, including itching of the eye. Methods of treating allergic conjunctivitis comprise administering to a human subject suffering from or susceptible to allergic conjunctivitis an effective amount of an ophthalmic composition described herein.

Typically, the compositions are administered as drops, with one drop of the composition being applied to an eye of the subject suffering from or susceptible to allergic conjunctivitis two times per day, although more or less of the composition may be used in more or less frequent doses depending on multiple factors, including the makeup of the particular composition.

The ophthalmic compositions may be formulated as single or multi dose units, with or without the use of a preservative, and may be manufactured by mixing the ingredients. The compositions may be packaged in single or multiple dosage forms, such as closed bottles, tubes, or other containers made from materials such as glass or plastic. In some embodiments, the packaging for the ophthalmic composition may be free or substantially free of antioxidant (e.g., as used in compositions described in U.S. Pat. Nos. 6,455,547 and 6,576,649).

### EXAMPLE

The invention will be further explained by the following illustrative example that is intended to be non-limiting.

Three different formulations of the ophthalmic compositions described herein were prepared with glycerol concentrations of 4%, 5%, and 6%. A ketotifen fumarate product marketed by Novartis Ophthalmics, Inc. (Duluth, Ga.) under the name Zaditorm was obtained for testing as a comparative product. Information concerning the ophthalmic compositions that were prepared and the comparative ketotifen fumarate product (as listed on the prescription information) is listed below in Table I.

TABLE I
Ophthalmic Compositions and Comparative Example

| | 4% Glycerol | 5% Glycerol | 6% Glycerol | Comparative |
|---|---|---|---|---|
| ketotifen fumarate | 0.0345% | 0.0345% | 0.0345% | 0.0345% |
| (ketotifen base) | (0.025%) | (0.025%) | (0.025%) | (0.025%) |
| Glycerol | 4.0% | 5.0% | 6.0% | not listed |
| benzalkonium chloride | 0.01% | 0.01% | 0.01% | 0.01% |
| pH | 5.68 | 5.73 | 5.74 | 4.4-5.8 |
| Osmolality (mOsm/Kg) | 454 | 561 | 689 | 210-300 |
| Other ingredients | purified water | purified water | purified water | purified water |
| | (balance), and | (balance), and | (balance), and | (balance), and |
| | sodium hydroxide | sodium hydroxide | sodium hydroxide | sodium hydroxide |
| | and/or hydrochloric | and/or hydrochloric | and/or hydrochloric | and/or hydrochloric |
| | acid to adjust pH | acid to adjust pH | acid to adjust pH | acid to adjust pH |

Two blinded tests were conducted in order to compare the comfort level of different solutions. In the first test, the comparative ketotifen fumarate product (i.e., Zaditor™) was tested against the 4% glycerol composition. Each human subject randomly received a drop of one of the two compositions in the right eye and received a drop of the other composition in the left eye, but the subjects were not informed of the identity of the compositions. The subject then indicated which eye was more comfortable. The blinded comparative test resulted in more subjects indicating that the 4% glycerol composition was more comfortable than the comparative ketotifen fumarate product.

In the second test, the 4% glycerol composition was tested against the 6% glycerol composition. Each of six human subjects randomly received a drop of one of the two compositions in the right eye and received a drop of the other composition in the left eye, but the subjects were not informed of the identity of the compositions. After receiving each drop, the subject indicated the comfort of the eye receiving the drop on a scale of 1-10, with 10 being the most comfortable. The results of the second test are listed below in Table II, which shows that the 6% glycerol composition was more comfortable than the 4% glycerol composition.

TABLE II
Comfort Indications of 4% Glycerol Composition
vs. 6% Glycerol Composition

| Composition | | | | | | | Average |
|---|---|---|---|---|---|---|---|
| 4% Glycerol | 7 | 5 | 8 | 6 | 4 | 4 | 5.66 |
| 6% Glycerol | 7 | 8 | 4 | 8 | 8 | 5 | 6.66 |

In conclusion, the results indicated that the 4% glycerol composition, which had an osmolality of 454 mOsm/Kg, was more comfortable in human eyes than the comparative ketotifen product, which has an osmolality listed on the corresponding prescription information of 210-300 mOsm/Kg. The results also indicated that the 6% glycerol composition, which had an osmolality of 689 mOsm/Kg, was more comfortable in human eyes than the 4% glycerol composition, which had an osmolality of 454 mOsm/Kg.

While the invention has been described in detail and with reference to specific embodiments thereof, it will be apparent to one skilled in the art that various changes and modifications can be made without departing from the spirit and scope of the invention.

What is claimed is:

1. An ophthalmic composition consisting essentially of:
(a) ketotifen or a ketotifen salt in a concentration of 0.01% to 0.05%;
(b) a non-ionic tonicity agent in a concentration such that the composition has an osmolality from 400 to 750 milliosmoles/Kg; and
(c) water.

2. The composition of claim 1, wherein the non-ionic tonicity agent is glycerol.

3. The composition of claim 1, wherein the composition includes a ketotifen salt and the ketotifen salt is ketotifen fumarate.

4. The composition of claim 3, wherein the concentration of the ketotifen fumarate is from 0.03% to 0.04%.

5. The composition of claim 1, wherein the composition has an osmolality from 425 to 700 milliosmoles/Kg.

6. The composition of claim 1, wherein the composition has an osmolality from 550 to 700 milliosmoles/Kg.

7. The composition of claim 1, wherein the composition has an osmolality from 600 to 700 milliosmoles/Kg.

8. The composition of claim 1, wherein the composition has an osmolality from 650 to 700 milliosmoles/Kg.

9. The composition of claim 1, wherein the concentration of the ketotifen or ketotifen salt is such that the concentration of ketotifen base in the composition is 0.02% to 0.03%.

10. The composition of claim 1, wherein the composition consists essentially of ketotifen fumarate in a concentration of 0.03% to 0.04%, glycerol in a concentration such that the composition has an osmolality from 650 to 700 milliosmoles/Kg, and water.

11. An ophthalmic composition consisting essentially of:
(a) ketotifen or a ketotifen salt in a concentration of 0.01% to 0.05%;
(b) glycerol in a concentration of 3.5% to 7%; and
(c) water.

12. The composition of claim 11, wherein the composition includes a ketotifen salt and the ketotifen salt is ketotifen fumarate.

13. The composition of claim 12, wherein the concentration of the ketotifen fumarate is from 0.03% to 0.04%.

14. The composition of claim 11, wherein the concentration of the glycerol is from 4.5% to 7%.

15. The composition of claim 11, wherein the concentration of the glycerol is from 5% to 7%.

16. The composition of claim 11, wherein the concentration of the glycerol is from 5.5% to 6.5%.

17. The composition of claim 11, wherein the concentration of the glycerol is from 5.75% to 6.25%.

18. The composition of claim 11, wherein the concentration of the ketotifen or ketotifen salt is such that the concentration of ketotifen base in the composition is 0.02% to 0.03%.

19. The composition of claim 11, wherein the composition consists essentially of ketotifen fumarate in a concentration of 0.03% to 0.04%, glycerol in a concentration of 5.75% to 6.25%, and water.

20. An ophthalmic composition consisting essentially of:
(a) ketotifen or a ketotifen salt in a concentration of 0.01% to 0.05%;
(b) glycerol in a concentration of greater than 3.5% such that the composition has an osmolality from 400 to 750 milliosmoles/Kg; and
(c) water.

21. The composition of claim 20, wherein the composition includes a ketotifen salt and the ketotifen salt is ketotifen fumarate.

22. The composition of claim 21, wherein the concentration of the ketotifen fumarate is from 0.03% to 0.04%.

23. The composition of claim 20, wherein the composition has an osmolality from 425 to 700 milliosmoles/Kg.

24. The composition of claim 20, wherein the composition has an osmolality from 550 to 700 milliosmoles/Kg.

25. The composition of claim 20, wherein the composition has an osmolality from 600 to 700 milliosmoles/Kg.

26. The composition of claim 20, wherein the composition has an osmolality from 650 to 700 milliosmoles/Kg.

27. The composition of claim 20, wherein the concentration of the glycerol is greater than 4.5%.

28. The composition of claim 20, wherein the concentration of the glycerol is greater than 5.5%.

29. The composition of claim 20, wherein the concentration of the glycerol is from 5% to 7%.

30. The composition of claim 20, wherein the concentration of the glycerol is from 5.5% to 6.5%.

31. The composition of claim 20, wherein the concentration of the ketotifen or ketotifen salt is such that the concentration of ketotifen base in the composition is 0.02% to 0.03%.

32. The composition of claim 20, wherein the composition consists essentially of ketotifen fumarate in a concentration of 0.03% to 0.04%, glycerol in a concentration of 5.5% to 6.5% such that the composition has an osmolality from 650 to 700 milliosmoles/Kg, and water.

33. An ophthalmic composition consisting essentially of:
(a) ketotifen fumarate in a concentration of 0.0345%;
(b) glycerol in a concentration of 5.75% to 6.25%;
(c) benzalkonium chloride in a concentration of 0.01%; and
(d) water;
wherein the pH of the composition is from 4.4 to 5.8.

34. A method of treating allergic conjunctivitis, comprising administering to a subject suffering from or susceptible to allergic conjunctivitis an effective amount of an ophthalmic composition consisting essentially of (a) ketotifen or a ketotifen salt in a concentration of 0.01% to 0.05%; (b) a non-ionic tonicity agent in a concentration such that the composition has an osmolality from 400 to 750 milliosmoles/Kg; and (c) water.

35. The method of claim 34, wherein the non-ionic tonicity agent of the composition is glycerol.

36. The method of claim 34, wherein the composition includes a ketotifen salt and the ketotifen salt is ketotifen fumarate.

37. The method of claim 36, wherein the concentration of the ketotifen fumarate is from 0.03% to 0.04%.

38. The method of claim 34, wherein the composition has an osmolality from 425 to 700 milliosmoles/Kg.

39. The method of claim 34, wherein the composition has an osmolality from 550 to 700 milliosmoles/Kg.

40. The method of claim 34, wherein the composition has an osmolality from 600 to 700 milliosmoles/Kg.

41. The method of claim 34, wherein the composition has an osmolality from 650 to 700 milliosmoles/Kg.

42. The method of claim 34, wherein the concentration of the ketotifen or ketotifen salt in the composition is such that the concentration of ketotifen base in the composition is 0.02% to 0.03%.

43. The method of claim 34, wherein the composition consists essentially of ketotifen fumarate in a concentration of 0.03% to 0.04%, glycerol in a concentration such that the composition has an osmolality from 650 to 700 milliosmoles/Kg, and water.

44. A method of treating allergic conjunctivitis, comprising administering to a subject suffering from or susceptible to allergic conjunctivitis an effective amount of an ophthalmic composition consisting essentially of (a) ketotifen or a ketotifen salt in a concentration of 0.01% to 0.05%; (b) glycerol in a concentration of 3.5% to 7%; and (c) water.

45. The method of claim 44, wherein the composition includes a ketotifen salt and the ketotifen salt is ketotifen fumarate.

46. The method of claim 45, wherein the concentration of the ketotifen fumarate is from 0.03% to 0.04%.

47. The method of claim 44, wherein the concentration of the glycerol in the composition is from 4.5% to 7%.

48. The method of claim 44, wherein the concentration of the glycerol in the composition is from 5% to 7%.

49. The method of claim 44, wherein the concentration of the glycerol in the composition is from 5.5% to 6.5%.

50. The method of claim 44, wherein the concentration of the glycerol in the composition is from 5.75% to 6.25%.

51. The method of claim 44, wherein the concentration of the ketotifen or ketotifen salt in the composition is such that the concentration of ketotifen base in the composition is 0.02% to 0.03%.

52. The method of claim 44, wherein the composition consists essentially of ketotifen fumarate in a concentration of 0.03% to 0.04%, glycerol in a concentration of 5.75% to 6.25%, and water.

53. A method of treating allergic conjunctivitis, comprising administering to a subject suffering from or susceptible to allergic conjunctivitis an effective amount of an ophthalmic composition consisting essentially of (a) ketotifen or a ketotifen salt in a concentration of 0.01% to 0.05%; (b) glycerol in a concentration of greater than 3.5% such that the composition has an osmolality from 400 to 750 milliosmoles/Kg; and (c) water.

54. The method of claim 53, wherein the composition includes a ketotifen salt and the ketotifen salt is ketotifen fumarate.

55. The method of claim 54, wherein the concentration of the ketotifen fumarate is from 0.03% to 0.04%.

56. The method of claim 53, wherein the composition has an osmolality from 425 to 700 milliosmoles/Kg.

57. The method of claim 53, wherein the composition has an osmolality from 550 to 700 milliosmoles/Kg.

58. The method of claim 53, wherein the composition has an osmolality from 600 to 700 milliosmoles/Kg.

59. The method of claim 53, wherein the composition has an osmolality from 650 to 700 milliosmoles/Kg.

60. The method of claim 53, wherein the concentration of the glycerol in the composition is greater than 4.5%.

61. The method of claim 53, wherein the concentration of the glycerol in the composition is greater than 5.5%.

62. The method of claim 53, wherein the concentration of the glycerol in the composition is from 5% to 7%.

63. The method of claim 53, wherein the concentration of the glycerol in the composition is from 5.5% to 6.5%.

64. The method of claim 53, wherein the concentration of the ketotifen or ketotifen salt in the composition is such that the concentration of ketotifen base in the composition is 0.02% to 0.03%.

65. The method of claim 53, wherein the composition consists essentially of ketotifen fumarate in a concentration of 0.03% to 0.04%, glycerol in a concentration of 5.5% to 6.5% such that the composition has an osmolality from 650 to 700 milliosmoles/Kg, and water.

66. A method of treating allergic conjunctivitis, comprising administering to a subject suffering from or susceptible to allergic conjunctivitis an effective amount of an ophthalmic composition consisting essentially of (a) ketotifen fumarate in a concentration of 0.0345%, (b) glycerol in a concentration of 5.75% to 6.25%, (c) benzalkonium chloride in a concentration of 0.01%, and (d) water, wherein the pH of the composition is from 4.4 to 5.8.

67. An ophthalmic composition comprising:
(a) ketotifen or a ketotifen salt in a concentration of 0.01% to 0.05%;
(b) a non-ionic tonicity agent in a concentration such that the composition has an osmolality from 400 to 750 milliosmoles/Kg; and
(c) water.

68. An ophthalmic composition comprising:
(a) ketotifen or a ketotifen salt in a concentration of 0.01% to 0.05%;
(b) glycerol in a concentration of 3.5% to 7%; and
(c) water.

69. An ophthalmic composition comprising:
(a) ketotifen or a ketotifen salt in a concentration of 0.01% to 0.05%;
(b) glycerol in a concentration of greater than 3.5% such that the composition has an osmolality from 400 to 750 milliosmoles/Kg; and
(c) water.

70. An ophthalmic composition comprising:
(a) ketotifen fumarate in a concentration of 0.0345%;
(b) glycerol in a concentration of 5.75% to 6.25%;
(c) benzalkonium chloride in a concentration of 0.01%; and
(d) water;
wherein the pH of the composition is from 4.4 to 5.8.
